(19) United States
(12) Patent Application Publication
(10) Pub. No.: US 2026/0245703 A1
(43) Pub. Date: Aug. 20, 2026

(54) APPARATUS AND METHOD FOR IN-VITRO FERTILIZATION TREATMENTS

(71) Applicant: BETTERFIT LTD., Ramat-Hasharon (IL)

(72) Inventor: Gon Shoham, Ramat-Hasharon (IL)

(21) Appl. No.: 18/994,781

(22) PCT Filed: Jul. 17, 2023
(86) PCT No.: PCT/IB2023/057264

§ 371 Date: Jan. 15, 2025

Related U.S. Application Data
(60) Provisional application No. 63/389,923, filed on Jul. 17, 2022.

(51) Int. Cl. G16H 30/40; G06T 7/00

(52) U.S. Cl. CPC G16H 30/40; G06T 7/0012; G06T 2207/10132; G06T 2207/20036; G06T 2207/20081; G06T 2207/30024

(57) ABSTRACT

Apparatus and methods are described including one or more computer processors (94, 96) configured, during a training stage, to receive one or more images of sperm that are selected for use in ICSI procedures, the images being acquired at the same time as the sperm for respective ICSI procedures are selected by a healthcare professional. Subsequently, inputs that are indicative of whether sperm that were selected for respective ICSI procedures gave rise to male or female embryos are received, and one or more computer processors (94, 96) analyze the images and the inputs that are indicative of whether sperm that were selected for respective ICSI procedures gave rise to male or female embryos, to identify features of sperm that are indicative of likelihoods of the sperm giving rise to male or female embryos. Other applications are also described.

### CROSS-REFERENCES TO RELATED APPLICATIONS

The present application claims priority from U.S. Provisional Patent Application 63/389,923 to Shoham, entitled “Apparatus and method for in-vitro fertilization procedures,” filed Jul. 17, 2022, which is incorporated herein by reference.

### FIELD OF EMBODIMENTS OF THE INVENTION

Some applications of the present invention generally relate to medical apparatus and methods. Specifically, some applications of the present invention relate to apparatus and methods for improving outcomes in in-vitro fertilization procedures.

### BACKGROUND

In vitro fertilization (hereinafter “IVF”) is a series of procedures that are used to assist with the conception of a child, typically in cases in which the parents have fertility and/or genetic conditions. During IVF, mature eggs are collected from ovaries and fertilized by sperm in a laboratory. One or more fertilized eggs (i.e., embryos) are then implanted in a uterus.

Sperm selection for use in an IVF treatment is typically performed as part of a procedure that is known as Intracytoplasmic Sperm Injection (hereinafter “ICSI”). In the ICSI procedure, an embryologist views many sperm through a microscope before selecting a specific sperm to be used for the fertilization. The sperm selection is typically based on criteria related to morphology and motility that were developed by the World Health Organization (referred to as the “WHO criteria,” see World Health Organization (1992) WHO Laboratory Manual for the Examination of Human Semen and Semen-Cervical Mucus Interaction, 3rd edn. Cambridge University Press, Cambridge, UK) and have been accepted by the medical community for many years. The selected sperm is immobilized by touching it with a pipette, before aspirating the sperm using the pipette. The aspirated sperm is then injected into the cytoplasm of an egg, while the egg is held by a holding tool. Subsequently, the fertilized egg (i.e., the embryo) is incubated as the cells divide, before being implanted in a uterus.

### SUMMARY

In accordance with some applications of the present invention, a sperm-selection optimization method is performed during ICSI procedures. Typically, during a training stage, a healthcare professional (e.g., an embryologist) views sperm via a microscope, selects a sperm in accordance with their usual criteria, and then immobilizes the sperm by touching the sperm with the pipette. In parallel the aforementioned step, one or more images of the selected sperm are acquired. Typically, the acquisition of the images is performed in such a manner that the usual workflow of the embryologist is unchanged and undisturbed. The embryologist continues with the ICSI procedure in the usual manner, by aspirating the sperm using the pipette, before injecting the aspirated sperm into the cytoplasm of an egg, while the egg is held by a holding tool. Typically, at a given time period after the injection of the sperm into the egg (for example, between 10 and 30 hours, e.g., between 15 and 18 hours, after the injection of the sperm into the egg), it is determined whether the egg was successfully fertilized. Typically, the embryologist (or a different healthcare professional) analyzes the egg to determine whether it has fertilized and inputs this information into a computer processor.

The data regarding whether the egg was successfully fertilized, together with the images that were acquired are received as inputs into a machine-learning prediction model that is run by one or more computer processors. Thus, the machine-learning prediction model receives both images of the sperm that was originally selected as well as data regarding whether or not the sperm successfully fertilized an egg. The machine-learning prediction model typically analyzes the images of the sperm that was originally selected as well as the input regarding whether or not the sperm successfully fertilized an egg, in order to detect features of the sperm that are indicative of sperm being of the type to successfully fertilize an egg or of the type that does not. Typically, the machine-learning prediction model analyzes the data from many procedures in which the preceding steps have been performed, such that the machine-learning prediction model has a large amount of data from which to determine features of the sperm that are indicative of sperm being of the type to successfully fertilize an egg or of the type not to do so. The one or more computer processors typically store the output of the machine-learning prediction model for use during the clinical-application stage, as described hereinbelow.

It is noted that the features that are identified within the data do not necessarily have any relationship with the WHO criteria for identifying sperm. Rather, in accordance with the above description, the machine-learning prediction model is configured to identify features of the sperm that are indicative of sperm being of the type to successfully fertilize an egg or of the type not to do so, based on real-world data regarding sperm that did actually successfully fertilize an egg or did not.

Typically, during a clinical-application stage, in a first step, a healthcare professional (e.g., the embryologist) views the sperm via a microscope, selects a sperm in accordance with their usual criteria, and then immobilizes the sperm by touching the sperm with the pipette. In parallel with the aforementioned step, one or more images of the selected sperm are acquired. Typically, the acquisition of the images is performed in such a manner that the usual workflow of the embryologist is unchanged and undisturbed. For some applications, the images that are acquired are received by a computer processor, for comparison with the features of sperm that were identified in the training stage as being indicative of whether a sperm is likely to fertilize an egg. Based on the comparison, the computer processor determines a likelihood of the sperm that was selected successfully fertilizing an egg. The computer processor typically generates an output to the embryologist indicating the likelihood of the sperm that was selected successfully fertilizing an egg and whether the sperm is therefore a good candidate for the remainder of the ICSI procedure. Typically, in response to the computer processor indicating that the selected sperm is a good candidate for the remainder of the ICSI procedure, the embryologist continues to perform the remainder of the ICSI procedure with the selected sperm. Further typically, in response to the computer processor indicating that the selected sperm is not a good candidate for the remainder of the ICSI procedure, the preceding steps are performed again with a different sperm.

For some applications, using generally similar techniques, mutatis mutandis, the computer processor analyzes the sperm that is selected by the embryologist to determine the likely sex of the resulting embryo. Typically in a first step, the embryologist views the sperm via a microscope, selects a sperm in accordance with their usual criteria, and then immobilizes the sperm by touching the sperm with the pipette. In parallel with this step, one or more images of the selected sperm are acquired. Typically, the embryologist continues with the ICSI procedure in the usual manner, by aspirating the sperm using the pipette, before injecting the aspirated sperm into the cytoplasm of an egg, while the egg is held by a holding tool. At a given time period after the fertilization of the egg at which it is possible to determine the sex of the resultant embryo, fetus, or baby, the sex of the resultant embryo, fetus, or baby is determined. Typically, the embryologist or a different healthcare professional analyzes the embryo fetus, or baby to determine the sex of the embryo and inputs this information into a computer processor. For example, the sex of the embryo is determined using preimplantation genetic testing, or it is determined via an ultrasound scan that is performed on the carrier of the embryo or fetus. For some applications, the sex of the resultant embryo or fetus is determined by performing amniocentesis or a placenta biopsy. For some applications, the sex of the resultant fetus is determined between 10 and 18 weeks (e.g., approximately 16 weeks) after the egg has been fertilized, typically by performing an ultrasound scan. For some applications, the sex determination is only performed after the baby is born. The scope of the present disclosure includes determining the sex of the resulting embryo, fetus, or baby by any means, whether during the pregnancy or after the baby is born. Subsequently, the data regarding the sex of the embryo, fetus, or baby together with the images that were acquired, are fed as inputs into a machine-learning prediction model that is run by one or more computer processors. Thus, the machine-learning prediction model receives both images of the sperm that was originally selected as well as data regarding the sex of the resultant embryo fetus, or baby. The machine-learning prediction model analyzes the images of the sperm that was originally selected as well as the input regarding the sex of the resultant embryo, fetus, or baby, in order to detect features of the sperm that are indicative of a likelihood of the sperm giving rise to a male or female embryo.

For some applications, using generally similar techniques, mutatis mutandis, the computer processor determines whether the sperm is likely to give rise to a viable or non-viable fetus, to give rise to a baby being born or not, to give rise to a healthy or an unhealthy baby, and/or to give rise to a baby having a given any medical condition, and generates an output accordingly.

For some applications, generally similar techniques to those described hereinabove are performed with respect to eggs that are selected for an IVF treatment (e.g., for an ICSI procedure of an IVF treatment). For some such applications, generally similar steps to those described with hereinabove are performed with reference to eggs, as an alternative, or in addition, to performing them with respect to sperm. Typically, during the training stage, features of eggs (e.g., morphological features of eggs) that are indicative of eggs giving rise to a successful fertilization are determined. Subsequently, in the clinical-application stage, in response to an egg being selected for use in the IVF treatment, the computer processor generates an output to the embryologist indicating a likelihood of the selected egg being successfully fertilized. For some applications, the machine-learning prediction model is trained with respect to combinations of sperm and eggs, such that it is trained to identify which combinations of features of sperm and eggs are likely to cause an egg to be successfully fertilized. Subsequently, during the clinical-application stage, the computer processor indicates a likelihood of a selected sperm successfully fertilizing a given egg, or vice versa (i.e., the computer processor indicates a likelihood of a selected egg being successfully fertilized by a given sperm). For some applications, generally similar techniques to those described hereinabove are performed with respect to an egg, in order to determine a likelihood of an egg (or a combination of a sperm and an egg) giving rise to a viable or non-viable fetus, giving rise to a born baby, giving rise to a healthy baby, and/or giving rise to a baby having one or more medical conditions.

There is therefore provided, in accordance with some applications of the present invention, apparatus including:
one or more computer processors configured:
during a training stage:
   to receive one or more images of sperm that are selected for use in ICSI procedures, the images being acquired at the same time as the sperm for respective ICSI procedures are selected by a healthcare professional,
   subsequently, receive inputs that are indicative of whether sperm that were selected for respective ICSI procedures gave rise to male or female embryos, and
   analyze the images and the inputs that are indicative of whether sperm that were selected for respective ICSI procedures gave rise to male or female embryos, to identify features of sperm that are indicative of likelihoods of the sperm giving rise to male or female embryos.

In some applications, the one or more computer processors are configured to receive inputs that are indicative of whether sperm that were selected for respective ICSI procedures gave rise to male or female embryos by receiving inputs based on preimplantation genetic testing performed on at least some embryos resulting from the ICSI procedures.

In some applications, the one or more computer processors are configured to receive inputs that are indicative of whether sperm that were selected for respective ICSI procedures gave rise to male or female embryos by receiving inputs based on ultrasound scans performed on at least some embryos or fetuses resulting from the ICSI procedures.

In some applications, the one or more computer processors are configured to receive inputs that are indicative of whether sperm that were selected for respective ICSI procedures gave rise to male or female embryos by receiving inputs based on amniocentesis performed on carriers of at least some of the embryos resulting from the ICSI procedures.

In some applications, the one or more computer processors are configured to receive inputs that are indicative of whether sperm that were selected for respective ICSI procedures gave rise to male or female embryos by receiving inputs based on placenta biopsies performed on carriers of at least some of the embryos resulting from the ICSI procedures.

In some applications, the one or more computer processors are configured to receive inputs that are indicative of whether sperm that were selected for respective ICSI procedures gave rise to male or female embryos by receiving inputs indicating whether born babies resulting from the ICSI procedures were male or female.

In some applications, the one or more computer processors are configured to receive inputs that are indicative of whether sperm that were selected for respective ICSI procedures gave rise to male or female embryos by receiving the inputs 10-18 weeks after the respective ICSI procedures.

In some applications, the one or more computer processors are configured to apply a transfer-learning algorithm to the identified features in order to:
analyze data relating to an additional set of sperm that were acquired under a different set of conditions from the sperm in the one more images, and
identify features in the data relating to the additional set of sperm that are indicative of likelihoods of sperm within the additional set of sperm giving rise to male or female embryos.

In some applications, the one or more computer processors are configured to receive one or more stationary images of the sperm that are selected for use in ICSI procedures and to identify morphological features of the sperm that are indicative of likelihoods of the sperm giving rise to male or female embryos by analyzing the one or more stationary images of the sperm.

In some applications, the one or more computer processors are configured to receive one or more video images of the sperm that are selected for use in ICSI procedures and to identify motility-related features of the sperm that are indicative of likelihoods of the sperm giving rise to male or female embryos by analyzing the one or more video images of the sperm.

In some applications, the one or more computer processors are further configured, during a clinical-application stage, to:
receive one or more images of a sperm that is selected for use in a current ICSI procedure, the images being acquired at the same time as the sperm for the current ICSI procedures is selected by a healthcare professional,
determine a likelihood of the selected sperm giving rise to a male or a female embryo by comparing features of the selected sperm to the features that were identified in the training stage, and
generate an output that is indicative of the likelihood of the selected sperm giving rise to a male or a female embryo.

In some applications, the one or more computer processors are further configured to:
receive inputs that are indicative of whether sperm that were selected for respective ICSI procedures gave rise to viable fetuses, and
analyze the images and the inputs that are indicative of whether sperm that were selected for respective ICSI procedures gave rise to viable fetuses, to identify features of sperm that are indicative of likelihoods of the sperm giving rise to viable fetuses.

In some applications, the one or more computer processors are further configured to:
receive inputs that are indicative of whether sperm that were selected for respective ICSI procedures gave rise to born babies, and
analyze the images and the inputs that are indicative of whether sperm that were selected for respective ICSI procedures gave rise to born babies, to identify features of sperm that are indicative of likelihoods of the sperm giving rise to born babies.

In some applications, the one or more computer processors are further configured to:
receive inputs that are indicative of whether sperm that were selected for respective ICSI procedures gave rise to healthy babies, and
analyze the images and the inputs that are indicative of whether sperm that were selected for respective ICSI procedures gave rise to healthy babies, to identify features of sperm that are indicative of likelihoods of the sperm giving rise to healthy babies.

In some applications, the one or more computer processors are further configured to:
receive inputs that are indicative of whether sperm that were selected for respective ICSI procedures gave rise to babies having a given medical condition, and
analyze the images and the inputs that are indicative of whether sperm that were selected for respective ICSI procedures gave rise to babies having the given medical condition, to identify features of sperm that are indicative of likelihoods of the sperm giving rise to babies having the given medical condition.

There is further provided, in accordance with some applications of the present invention, a method including:
using one or more computer processors, during a training stage:
   receiving one or more images of sperm that are selected for use in ICSI procedures, the images being acquired at the same time as the sperm for respective ICSI procedures are selected by a healthcare professional;
   subsequently, receiving inputs that are indicative of whether sperm that were selected for respective ICSI procedures gave rise to male or female embryos; and
   analyzing the images and the inputs that are indicative of whether sperm that were selected for respective ICSI procedures gave rise to male or female embryos, to identify features of sperm that are indicative of likelihoods of the sperm giving rise to male or female embryos.

In some applications, receiving inputs that are indicative of whether sperm that were selected for respective ICSI procedures gave rise to male or female embryos includes receiving inputs based on preimplantation genetic testing performed on at least some embryos resulting from the ICSI procedures.

In some applications, receiving inputs that are indicative of whether sperm that were selected for respective ICSI procedures gave rise to male or female embryos includes receiving inputs based on ultrasound scans performed on at least some embryos or fetuses resulting from the ICSI procedures.

In some applications, receiving inputs that are indicative of whether sperm that were selected for respective ICSI procedures gave rise to male or female embryos includes receiving inputs based on amniocentesis performed on carriers of at least some of the embryos resulting from the ICSI procedures.

In some applications, receiving inputs that are indicative of whether sperm that were selected for respective ICSI procedures gave rise to male or female embryos includes receiving inputs based on placenta biopsies performed on carriers of at least some of the embryos resulting from the ICSI procedures.

In some applications, receiving inputs that are indicative of whether sperm that were selected for respective ICSI procedures gave rise to male or female embryos includes receiving inputs indicating whether born babies resulting from the ICSI procedures were male or female.

In some applications, receiving inputs that are indicative of whether sperm that were selected for respective ICSI procedures gave rise to male or female embryos includes receiving the inputs 10-18 weeks after the respective ICSI procedures.

In some applications, the method further includes applying a transfer-learning algorithm to the identified features in order to:
analyze data relating to an additional set of sperm that were acquired under a different set of conditions from the sperm in the one more images, and
identify features in the data relating to the additional set of sperm that are indicative of likelihoods of sperm within the additional set of sperm giving rise to male or female embryos.

In some applications, receiving one or more images of sperm that are selected for use in ICSI procedures includes receiving one or more stationary images of the sperm that are selected for use in ICSI procedures, the method including identifying morphological features of the sperm that are indicative of likelihoods of the sperm giving rise to male or female embryos by analyzing the one or more stationary images of the sperm.

In some applications, receiving one or more images of sperm that are selected for use in ICSI procedures includes receiving one or more video images of the sperm that are selected for use in ICSI procedures, the method including identifying motility-related features of the sperm that are indicative of likelihoods of the sperm giving rise to male or female embryos by analyzing the one or more video images of the sperm.

In some applications, the method further includes, during a clinical-application stage:
receiving one or more images of a sperm that is selected for use in a current ICSI procedure, the images being acquired at the same time as the sperm for the current ICSI procedures is selected by a healthcare professional;
determining a likelihood of the selected sperm giving rise to a male or a female embryo by comparing features of the selected sperm to the features that were identified in the training stage; and
generating an output that is indicative of the likelihood of the selected sperm giving rise to a male or a female embryo.

In some applications, the method further includes:
receiving inputs that are indicative of whether sperm that were selected for respective ICSI procedures gave rise to viable fetuses, and
analyzing the images and the inputs that are indicative of whether sperm that were selected for respective ICSI procedures gave rise to viable fetuses, to identify features of sperm that are indicative of likelihoods of the sperm giving rise to viable fetuses.

In some applications, the method further includes:
receiving inputs that are indicative of whether sperm that were selected for respective ICSI procedures gave rise to born babies, and
analyzing the images and the inputs that are indicative of whether sperm that were selected for respective ICSI procedures gave rise to born babies, to identify features of sperm that are indicative of likelihoods of the sperm giving rise to born babies.

In some applications, the method further includes:
receiving inputs that are indicative of whether sperm that were selected for respective ICSI procedures gave rise to healthy babies, and
analyzing the images and the inputs that are indicative of whether sperm that were selected for respective ICSI procedures gave rise to healthy babies, to identify features of sperm that are indicative of likelihoods of the sperm giving rise to healthy babies.

In some applications, the method further includes:
receiving inputs that are indicative of whether sperm that were selected for respective ICSI procedures gave rise to babies having a given medical condition, and
analyzing the images and the inputs that are indicative of whether sperm that were selected for respective ICSI procedures gave rise to babies having the given medical condition, to identify features of sperm that are indicative of likelihoods of the sperm giving rise to babies having the given medical condition.

There is further provided, in accordance with some applications of the present invention, a computer software product including a tangible non-transitory computer-readable medium in which program instructions are stored, which instructions, when read by one or more computer processors, cause the one or more computer processors, during a training stage:
to receive one or more images of sperm that are selected for use in ICSI procedures, the images being acquired at the same time as the sperm for respective ICSI procedures are selected by a healthcare professional,
subsequently, receive inputs that are indicative of whether sperm that were selected for respective ICSI procedures gave rise to male or female embryos, and
analyze the images and the inputs that are indicative of whether sperm that were selected for respective ICSI procedures gave rise to male or female embryos, to identify features of sperm that are indicative of likelihoods of the sperm giving rise to male or female embryos.

In some applications, the computer software product is configured to cause the one or more computer processors to receive inputs that are indicative of whether sperm that were selected for respective ICSI procedures gave rise to male or female embryos by receiving inputs based on preimplantation genetic testing performed on at least some embryos resulting from the ICSI procedures.

In some applications, the computer software product is configured to cause the one or more computer processors to receive inputs that are indicative of whether sperm that were selected for respective ICSI procedures gave rise to male or female embryos by receiving inputs based on ultrasound scans performed on at least some embryos or fetuses resulting from the ICSI procedures.

In some applications, the computer software product is configured to cause the one or more computer processors to receive inputs that are indicative of whether sperm that were selected for respective ICSI procedures gave rise to male or female embryos by receiving inputs based on amniocentesis performed on carriers of at least some of the embryos resulting from the ICSI procedures.

In some applications, the computer software product is configured to cause the one or more computer processors to receive inputs that are indicative of whether sperm that were selected for respective ICSI procedures gave rise to male or female embryos by receiving inputs based on placenta biopsies performed on carriers of at least some of the embryos resulting from the ICSI procedures.

In some applications, the computer software product is configured to cause the one or more computer processors to receive inputs that are indicative of whether sperm that were selected for respective ICSI procedures gave rise to male or female embryos by receiving inputs indicating whether born babies resulting from the ICSI procedures were male or female.

In some applications, the computer software product is configured to cause the one or more computer processors to receive inputs that are indicative of whether sperm that were selected for respective ICSI procedures gave rise to male or female embryos by receiving the inputs 10-18 weeks after the respective ICSI procedures.

In some applications, the computer software product is configured to cause the one or more computer processors to apply a transfer-learning algorithm to the identified features in order to:
analyze data relating to an additional set of sperm that were acquired under a different set of conditions from the sperm in the one more images, and
identify features in the data relating to the additional set of sperm that are indicative of likelihoods of sperm within the additional set of sperm giving rise to male or female embryos.

In some applications, the computer software product is configured to cause the one or more computer processors to receive one or more stationary images of the sperm that are selected for use in ICSI procedures and to identify morphological features of the sperm that are indicative of likelihoods of the sperm giving rise to male or female embryos by analyzing the one or more stationary images of the sperm.

In some applications, the computer software product is configured to cause the one or more computer processors to receive one or more video images of the sperm that are selected for use in ICSI procedures and to identify motility-related features of the sperm that are indicative of likelihoods of the sperm giving rise to male or female embryos by analyzing the one or more video images of the sperm.

In some applications, the computer software product is configured to cause the one or more computer processors, during a clinical-application stage, to:
receive one or more images of a sperm that is selected for use in a current ICSI procedure, the images being acquired at the same time as the sperm for the current ICSI procedures is selected by a healthcare professional,
determine a likelihood of the selected sperm giving rise to a male or a female embryo by comparing features of the selected sperm to the features that were identified in the training stage, and
generate an output that is indicative of the likelihood of the selected sperm giving rise to a male or a female embryo.

In some applications, the computer software product is configured to cause the one or more computer processors to:
receive inputs that are indicative of whether sperm that were selected for respective ICSI procedures gave rise to viable fetuses, and
analyze the images and the inputs that are indicative of whether sperm that were selected for respective ICSI procedures gave rise to viable fetuses, to identify features of sperm that are indicative of likelihoods of the sperm giving rise to viable fetuses.

In some applications, the computer software product is configured to cause the one or more computer processors to:
receive inputs that are indicative of whether sperm that were selected for respective ICSI procedures gave rise to born babies, and
analyze the images and the inputs that are indicative of whether sperm that were selected for respective ICSI procedures gave rise to born babies, to identify features of sperm that are indicative of likelihoods of the sperm giving rise to born babies.

In some applications, the computer software product is configured to cause the one or more computer processors to:
receive inputs that are indicative of whether sperm that were selected for respective ICSI procedures gave rise to healthy babies, and
analyze the images and the inputs that are indicative of whether sperm that were selected for respective ICSI procedures gave rise to healthy babies, to identify features of sperm that are indicative of likelihoods of the sperm giving rise to healthy babies.

In some applications, the computer software product is configured to cause the one or more computer processors to:
receive inputs that are indicative of whether sperm that were selected for respective ICSI procedures gave rise to babies having a given medical condition, and
analyze the images and the inputs that are indicative of whether sperm that were selected for respective ICSI procedures gave rise to babies having the given medical condition, to identify features of sperm that are indicative of likelihoods of the sperm giving rise to babies having the given medical condition.

There is further provided, in accordance with some applications of the present invention, apparatus including:
using one or more computer processors configured, during a training stage:
   to receive one or more images of sperm that are selected for use in ICSI procedures, the images being acquired at the same time as the sperm for respective ICSI procedures are selected by a healthcare professional,
   subsequently, to receive inputs that are indicative of whether sperm that were selected for respective ICSI procedures resulted in fertilizations of eggs, and
   analyze the images and the inputs that are indicative of whether sperm that were selected for respective ICSI procedures resulted in fertilizations of eggs, to identify features of sperm that are indicative of likelihoods of the sperm resulting in successful fertilizations.

In some applications, the one or more computer processors are configured to apply a transfer-learning algorithm to the identified features in order to:
analyze data relating to an additional set of sperm that were acquired under a different set of conditions from the sperm in the one more images, and
identify features in the data relating to the additional set of sperm that are indicative of likelihoods of sperm within the additional set of sperm resulting in successful fertilizations.

In some applications, the one or more computer processors are configured to receive one or more stationary images of the sperm that are selected for use in ICSI procedures and to identify morphological features of the sperm that are indicative of likelihoods of the sperm resulting in successful fertilizations by analyzing the one or more stationary images of the sperm.

In some applications, the one or more computer processors are configured to receive one or more video images of the sperm that are selected for use in ICSI procedures and to identify motility-related features of the sperm that are indicative of likelihoods of the sperm resulting in successful fertilizations by analyzing the one or more video images of the sperm.

In some applications, the one or more computer processors are further configured to:
receive inputs that are indicative of whether sperm that were selected for respective ICSI procedures gave rise to male or female embryos, and
analyze the images and the inputs that are indicative of whether sperm that were selected for respective ICSI procedures gave rise to male or female embryos, to identify features of sperm that are indicative of likelihoods of the sperm giving rise to male or female embryos.

In some applications, the one or more computer processors are further configured to:
receive inputs that are indicative of whether sperm that were selected for respective ICSI procedures gave rise to viable fetuses, and
analyze the images and the inputs that are indicative of whether sperm that were selected for respective ICSI procedures gave rise to viable fetuses, to identify features of sperm that are indicative of likelihoods of the sperm giving rise to viable fetuses.

In some applications, the one or more computer processors are further configured to:
receive inputs that are indicative of whether sperm that were selected for respective ICSI procedures gave rise to born babies, and
analyze the images and the inputs that are indicative of whether sperm that were selected for respective ICSI procedures gave rise to born babies, to identify features of sperm that are indicative of likelihoods of the sperm giving rise to born babies.

In some applications, the one or more computer processors are further configured to:
receive inputs that are indicative of whether sperm that were selected for respective ICSI procedures gave rise to healthy babies, and
analyze the images and the inputs that are indicative of whether sperm that were selected for respective ICSI procedures gave rise to healthy babies, to identify features of sperm that are indicative of likelihoods of the sperm giving rise to healthy babies.

In some applications, the one or more computer processors are further configured to:
receive inputs that are indicative of whether sperm that were selected for respective ICSI procedures gave rise to babies having a given medical condition, and
analyze the images and the inputs that are indicative of whether sperm that were selected for respective ICSI procedures gave rise to babies having the given medical condition, to identify features of sperm that are indicative of likelihoods of the sperm giving rise to babies having the given medical condition.

In some applications, the one or more computer processors are further configured to:
receive inputs that are indicative of whether sperm that were selected for respective ICSI procedures gave rise to male or female embryos, and
analyze the images and the inputs that are indicative of whether sperm that were selected for respective ICSI procedures gave rise to male or female embryos, to identify features of sperm that are indicative of likelihoods of the sperm giving rise to male or female embryos.

In some applications, the one or more computer processors are further configured during a clinical-application stage, to:
receive one or more images of a sperm that is selected for use in a current ICSI procedure, the images being acquired at the same time as the sperm for the current ICSI procedures is selected by a healthcare professional,
determine a likelihood of the selected sperm resulting in a successful fertilization by comparing features of the selected sperm to the features that were identified in the training stage, and
generate an output that is indicative of the likelihood of the selected sperm resulting in a successful fertilization.

In some applications:
during the training stage, the one or more computer processors are configured to identify features of sperm that are indicative of whether the sperm will result in successful fertilizations of eggs having given features, and
during the clinical-application stage, the one or more computer processors are configured to determine a likelihood of the selected sperm resulting in a successful fertilization of a given egg by comparing features of the selected sperm to the features that were identified in the training stage.

In some applications, the one or more computer processors are configured to identify features of sperm that are indicative of whether the sperm will result in successful fertilizations of eggs having given features.

In some applications, the one or more computer processors are configured to identify features of sperm that are indicative of whether the sperm will result in successful fertilizations of eggs having given morphological features.

There is further provided, in accordance with some applications of the present invention, a method including:
using one or more computer processors, during a training stage:
   receiving one or more images of sperm that are selected for use in ICSI procedures, the images being acquired at the same time as the sperm for respective ICSI procedures are selected by a healthcare professional;
   subsequently, receiving inputs that are indicative of whether sperm that were selected for respective ICSI procedures resulted in fertilizations of eggs; and
   analyzing the images and the inputs that are indicative of whether sperm that were selected for respective ICSI procedures resulted in fertilizations of eggs, to identify features of sperm that are indicative of likelihoods of the sperm resulting in successful fertilizations.

In some applications, the method further includes applying a transfer-learning algorithm to the identified features in order to:
analyze data relating to an additional set of sperm that were acquired under a different set of conditions from the sperm in the one more images, and
identify features in the data relating to the additional set of sperm that are indicative of likelihoods of sperm within the additional set of sperm resulting in successful fertilizations.

In some applications, receiving one or more images of sperm that are selected for use in ICSI procedures includes receiving one or more stationary images of the sperm that are selected for use in ICSI procedures, the method including identifying morphological features of the sperm by analyzing the one or more stationary images of the sperm.

In some applications, receiving one or more images of sperm that are selected for use in ICSI procedures includes receiving one or more video images of the sperm that are selected for use in ICSI procedures, the method including identifying motility-related features of the sperm by analyzing the one or more video images of the sperm.

In some applications, the method further includes:
receiving inputs that are indicative of whether sperm that were selected for respective ICSI procedures gave rise to male or female embryos, and
analyzing the images and the inputs that are indicative of whether sperm that were selected for respective ICSI procedures gave rise to male or female embryos, to identify features of sperm that are indicative of likelihoods of the sperm giving rise to male or female embryos.

In some applications, the method further includes:
receiving inputs that are indicative of whether sperm that were selected for respective ICSI procedures gave rise to viable fetuses, and
analyzing the images and the inputs that are indicative of whether sperm that were selected for respective ICSI procedures gave rise to viable fetuses, to identify features of sperm that are indicative of likelihoods of the sperm giving rise to viable fetuses.

In some applications, the method further includes:
receiving inputs that are indicative of whether sperm that were selected for respective ICSI procedures gave rise to born babies, and
analyzing the images and the inputs that are indicative of whether sperm that were selected for respective ICSI procedures gave rise to born babies, to identify features of sperm that are indicative of likelihoods of the sperm giving rise to born babies.

In some applications, the method further includes:
receiving inputs that are indicative of whether sperm that were selected for respective ICSI procedures gave rise to healthy babies, and
analyzing the images and the inputs that are indicative of whether sperm that were selected for respective ICSI procedures gave rise to healthy babies, to identify features of sperm that are indicative of likelihoods of the sperm giving rise to healthy babies.

In some applications, the method further includes:
receiving inputs that are indicative of whether sperm that were selected for respective ICSI procedures gave rise to babies having a given medical condition, and
analyzing the images and the inputs that are indicative of whether sperm that were selected for respective ICSI procedures gave rise to babies having the given medical condition, to identify features of sperm that are indicative of likelihoods of the sperm giving rise to babies having the given medical condition.

In some applications, the method further includes:
receiving inputs that are indicative of whether sperm that were selected for respective ICSI procedures gave rise to male or female embryos, and
analyzing the images and the inputs that are indicative of whether sperm that were selected for respective ICSI procedures gave rise to male or female embryos, to identify features of sperm that are indicative of likelihoods of the sperm giving rise to male or female embryos.

In some applications, the method further includes, during a clinical-application stage:
receiving one or more images of a sperm that is selected for use in a current ICSI procedure, the images being acquired at the same time as the sperm for the current ICSI procedures is selected by a healthcare professional;
determining a likelihood of the selected sperm resulting in a successful fertilization by comparing features of the selected sperm to the features that were identified in the training stage; and
generating an output that is indicative of the likelihood of the selected sperm resulting in a successful fertilization.

In some applications, the method includes:
during the training stage, identifying features of sperm that are indicative of whether the sperm will result in successful fertilizations of eggs having given features, and
during the clinical-application stage, determining a likelihood of the selected sperm resulting in a successful fertilization of a given egg by comparing features of the selected sperm to the features that were identified in the training stage.

In some applications, the method includes identifying features of sperm that are indicative of whether the sperm will result in successful fertilizations of eggs having given features.

In some applications, identifying features of sperm that are indicative of whether the sperm will result in successful fertilizations of eggs having given features includes identifying features of sperm that are indicative of whether the sperm will result in successful fertilizations of eggs having given morphological features.

There is further provided, in accordance with some applications of the present invention, a computer software product including a tangible non-transitory computer-readable medium in which program instructions are stored, which instructions, when read by one or more computer processors, cause the one or more computer processors, during a training stage:
to receive one or more images of sperm that are selected for use in ICSI procedures, the images being acquired at the same time as the sperm for respective ICSI procedures are selected by a healthcare professional,
   subsequently, to receive inputs that are indicative of whether sperm that were selected for respective ICSI procedures resulted in fertilizations of eggs, and
   analyze the images and the inputs that are indicative of whether sperm that were selected for respective ICSI procedures resulted in fertilizations of eggs, to identify features of sperm that are indicative of likelihoods of the sperm resulting in successful fertilizations.

In some applications, the computer software product is configured to cause the one or more computer processors to apply a transfer-learning algorithm to the identified features in order to:
analyze data relating to an additional set of sperm that were acquired under a different set of conditions from the sperm in the one more images, and
identify features in the data relating to the additional set of sperm that are indicative of likelihoods of sperm within the additional set of sperm resulting in successful fertilizations.

In some applications, the computer software product is configured to cause the one or more computer processors to receive one or more stationary images of the sperm that are selected for use in ICSI procedures and to derive morphological features of the sperm by analyzing the one or more stationary images of the sperm.

In some applications, the computer software product is configured to cause the one or more computer processors to receive one or more video images of the sperm that are selected for use in ICSI procedures and to derive motility-related features of the sperm by analyzing the one or more video images of the sperm.

In some applications, the computer software product is configured to cause the one or more computer processors to:
receive inputs that are indicative of whether sperm that were selected for respective ICSI procedures gave rise to male or female embryos, and
analyze the images and the inputs that are indicative of whether sperm that were selected for respective ICSI procedures gave rise to male or female embryos, to identify features of sperm that are indicative of likelihoods of the sperm giving rise to male or female embryos.

In some applications, the computer software product is configured to cause the one or more computer processors:
receive inputs that are indicative of whether sperm that were selected for respective ICSI procedures gave rise to viable fetuses, and
analyze the images and the inputs that are indicative of whether sperm that were selected for respective ICSI procedures gave rise to viable fetuses, to identify features of sperm that are indicative of likelihoods of the sperm giving rise to viable fetuses.

In some applications, the computer software product is configured to cause the one or more computer processors:
receive inputs that are indicative of whether sperm that were selected for respective ICSI procedures gave rise to born babies, and
analyze the images and the inputs that are indicative of whether sperm that were selected for respective ICSI procedures gave rise to born babies, to identify features of sperm that are indicative of likelihoods of the sperm giving rise to born babies.

In some applications, the computer software product is configured to cause the one or more computer processors:
receive inputs that are indicative of whether sperm that were selected for respective ICSI procedures gave rise to healthy babies, and
analyze the images and the inputs that are indicative of whether sperm that were selected for respective ICSI procedures gave rise to healthy babies, to identify features of sperm that are indicative of likelihoods of the sperm giving rise to healthy babies.

In some applications, the computer software product is configured to cause the one or more computer processors:
receive inputs that are indicative of whether sperm that were selected for respective ICSI procedures gave rise to babies having a given medical condition, and
analyze the images and the inputs that are indicative of whether sperm that were selected for respective ICSI procedures gave rise to babies having the given medical condition, to identify features of sperm that are indicative of likelihoods of the sperm giving rise to babies having the given medical condition.

In some applications, the computer software product is configured to cause the one or more computer processors:
receive inputs that are indicative of whether sperm that were selected for respective ICSI procedures gave rise to male or female embryos, and
analyze the images and the inputs that are indicative of whether sperm that were selected for respective ICSI procedures gave rise to male or female embryos, to identify features of sperm that are indicative of likelihoods of the sperm giving rise to male or female embryos.

In some applications, the computer software product is configured to cause the one or more computer processors, during a clinical-application stage, to:
receive one or more images of a sperm that is selected for use in a current ICSI procedure, the images being acquired at the same time as the sperm for the current ICSI procedures is selected by a healthcare professional,
determine a likelihood of the selected sperm resulting in a successful fertilization by comparing features of the selected sperm to the features that were identified in the training stage, and
generate an output that is indicative of the likelihood of the selected sperm resulting in a successful fertilization.

In some applications, the computer software product is configured to cause the one or more computer processors:
during the training stage, to identify features of sperm that are indicative of whether the sperm will result in successful fertilizations of eggs having given features, and
during the clinical-application stage, to determine a likelihood of the selected sperm resulting in a successful fertilization of a given egg by comparing features of the selected sperm to the features that were identified in the training stage.

In some applications, the computer software product is configured to cause the one or more computer processors to identify features of sperm that are indicative of whether the sperm will result in successful fertilizations of eggs having given features.

In some applications, the computer software product is configured to cause the one or more computer processors to identify features of sperm that are indicative of whether the sperm will result in successful fertilizations of eggs having given morphological features.

There is further provided, in accordance with some applications of the present invention, apparatus including:
a microscope configured to display images of sperm to a healthcare professional, to facilitate the selection of a sperm by the healthcare professional for use in an ICSI procedure, the microscope including an auxiliary optical output configured to output one or more auxiliary images of the selected sperm; and
one or more computer processors configured to:
   receive the one or more auxiliary images of the selected sperm,
   determine a likelihood of the selected sperm resulting in a successful fertilization, and
   generate an output that is indicative of the likelihood of the selected sperm resulting in a successful fertilization.

There is further provided, in accordance with some applications of the present invention, apparatus including:
a microscope configured to display images of sperm to a healthcare professional, to facilitate the selection of a sperm by the healthcare professional for use in an ICSI procedure, the microscope including an auxiliary optical output configured to output one or more auxiliary images of the selected sperm; and
one or more computer processors configured to:
   receive the one or more auxiliary images of the selected sperm,
   determine a likelihood of the selected sperm giving rise to a male or a female embryo, and
   generate an output that is indicative of the likelihood of the selected sperm giving rise to a male or a female embryo.

There is further provided, in accordance with some applications of the present invention, apparatus including:
using one or more computer processors configured:
during a training stage:
   to receive one or more images of eggs that are selected for use in ICSI procedures, the images being acquired at the same time as the eggs for respective ICSI procedures are selected by a healthcare professional,
   subsequently, to receive inputs that are indicative of whether eggs that were selected for respective ICSI procedures resulted in fertilizations, and
   analyze the images and the inputs that are indicative of whether eggs that were selected for respective ICSI procedures resulted in fertilizations, to identify features of eggs that are indicative of whether the eggs will result in successful fertilizations.

In some applications, the one or more processors are configured to identify morphological features of eggs that are indicative of whether the eggs will result in successful fertilizations.

In some applications, the one or more computer processors are further configured, during a clinical-application stage, to:
receive one or more images of an egg that is selected for use in a current ICSI procedure, the images being acquired at the same time as the egg for the current ICSI procedures is selected by a healthcare professional,
determine a likelihood of the selected egg resulting in a successful fertilization by comparing features of the selected egg to the features that were identified in the training stage, and
generate an output that is indicative of the likelihood of the selected egg resulting in a successful fertilization.

In some applications:
during the training stage, the one or more computer processors are configured to identify to identify features of eggs that are indicative of whether the eggs will result in successful fertilizations by sperm having given features, and
during the clinical-application stage, the one or more computer processors are configured to determine a likelihood of the selected egg resulting in a successful fertilization by a given sperm, by comparing features of the selected egg to the features that were identified in the training stage.

The present invention will be more fully understood from the following detailed description of embodiments thereof, taken together with the drawings, in which:

### BRIEF DESCRIPTION OF THE DRAWINGS

FIG. 1 is an example of an image that an embryologist views via a microscope during the sperm selection stage of an ICSI procedure, in accordance with some applications of the present invention;

FIGS. 2A and 2B are flowcharts showing steps of a sperm-selection optimization method that is performed during ICSI procedures, respectively during a training stage and during a clinical-application stage, in accordance with some applications of the present invention;

FIGS. 3A and 3B are flowcharts showing steps of a sex-selection method that is performed during ICSI procedures, respectively during a training stage and during a clinical-application stage, in accordance with some applications of the present invention; and

FIG. 4 is a photograph showing an example of a microscope with an auxiliary optical output, in accordance with some applications of the present invention.

### DETAILED DESCRIPTION OF EMBODIMENTS

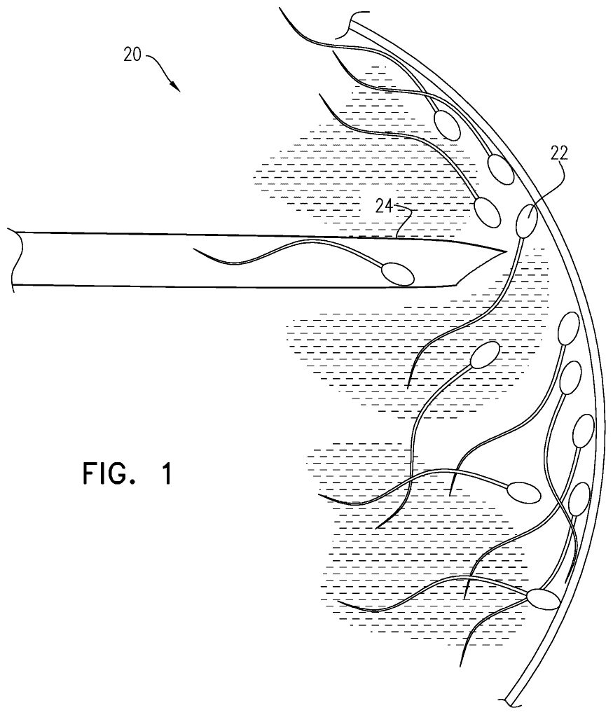

Reference is now made to FIG. 1, which is an example of an image 20 that an embryologist views via a microscope during the sperm selection stage of an ICSI procedure, in accordance with some applications of the present invention. The embryologist typically sees many sperm 22. As described hereinabove in the Background, the embryologist typically selects a specific sperm to be used for the fertilization based on criteria related to morphology and motility that were developed by the World Health Organization (the “WHO criteria”) and have been accepted by the medical community for many years. The selected sperm is immobilized by touching it with a pipette 24, before aspirating the sperm using the pipette. FIG. 1 shows a sperm that has been selected and aspirated into the pipette. It is noted that the scale of the image that is shown in FIG. 1 is much larger than the scale that the embryologist typically sees via the microscope during the ICSI procedure.

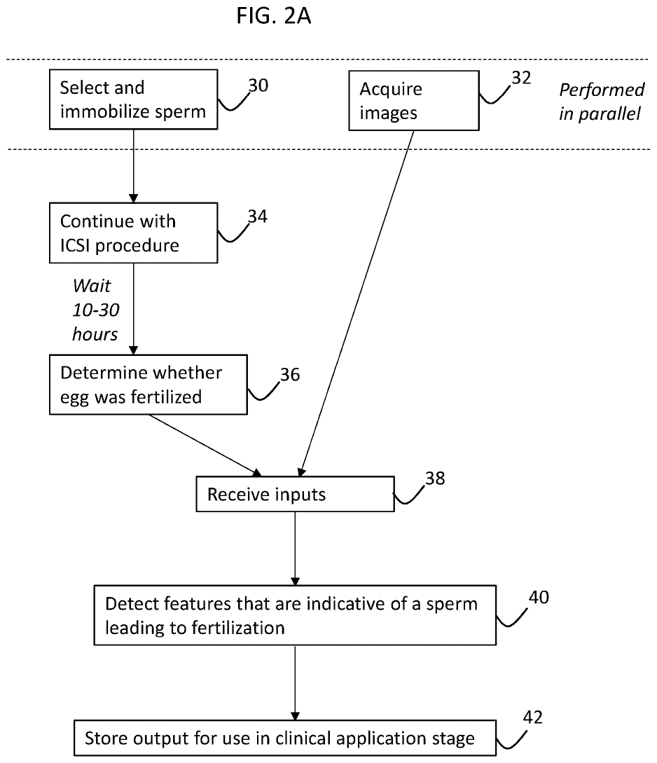

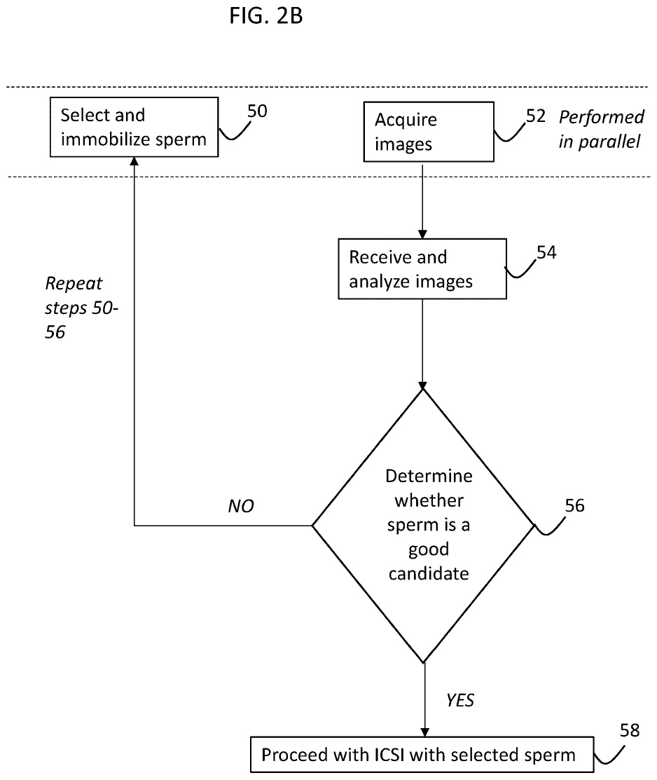

Reference is now made to FIG. 2A which is a flowchart showing steps of a sperm-selection optimization method that is performed during ICSI procedures during a training stage, in accordance with some applications of the present invention. Typically in a first step 30, a healthcare professional (e.g., the embryologist) views the sperm via a microscope, selects a sperm in accordance with their usual criteria, and then immobilizes the sperm by touching the sperm with the pipette. In parallel with step 30, one or more images of the selected sperm are acquired, in step 32. For some applications, in order acquire the images of the selected sperm in step 32, the microscope includes an auxiliary optical output (in addition to the first optical output via which the images are shown to the embryologist in real time). The images that are acquired in step 32 are typically sent to a computer processor for further analysis as described in further detail hereinbelow. Typically, the images that are acquired in step 32 are of a higher quality than the images that are shown to the embryologist, and the auxiliary optical output has a higher resolution than that of the first optical output. As described above, step 32 is typically performed in parallel with step 30. Further typically, the acquisition of the images in step 32 is performed in such a manner that the usual workflow of the embryologist is unchanged and undisturbed.

Typically, both the first optical output (via which the images are shown to the embryologist in real time) and the auxiliary optical output (via which images are outputted for further analysis by a computer processor) receive (and output) optical images that are acquired using cameras, e.g., CMOS cameras. For some applications, both of the cameras acquire (and output) images within the visible light spectral range. For some applications, the first optical output receives (and outputs) images within the visible light spectral range and the auxiliary optical output receives (and outputs) images in an alternative or an additional spectral range, e.g., in the short wave infrared (SWIR) spectral range.

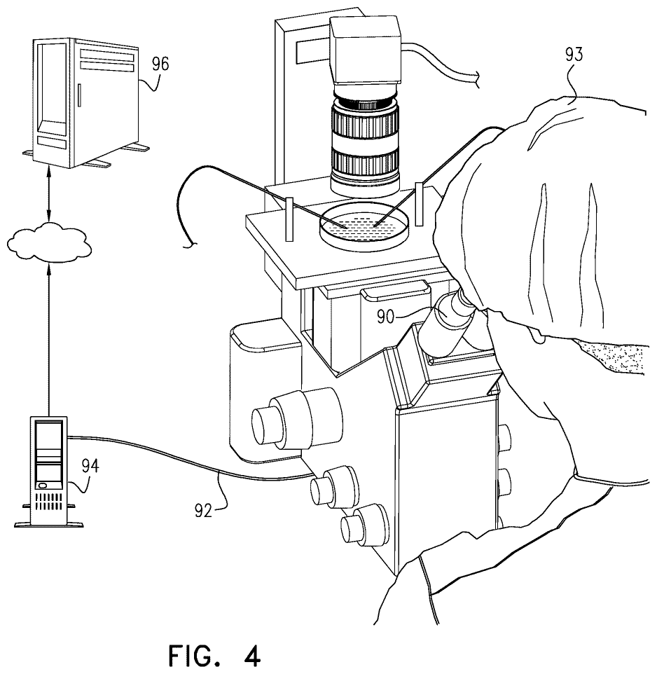

In step 34, the embryologist continues with the ICSI procedure in the usual manner, by aspirating the sperm using the pipette, before injecting the aspirated sperm into the cytoplasm of an egg, while the egg is held by a holding tool. In step 36, at a given time period after the injection of the sperm into the egg (for example, between 10 and 30 hours, e.g., between 15 and 18 hours, after the injection of the sperm into the egg), it is determined whether the egg was successfully fertilized. Typically, the embryologist (or a different healthcare professional) analyzes the egg to determine whether it has fertilized and inputs this information into a computer processor. In step 38, the data regarding whether the egg was successfully fertilized, together with the images that were acquired in step 32 are received as inputs into a machine-learning prediction model that is run by one or more computer processors (which are typically in communication with each other via a network, e.g., computer processors 94 and 96 described hereinbelow with reference to FIG. 4). Thus, the machine-learning prediction model receives both images of the sperm that was originally selected as well as data regarding whether or not the sperm successfully fertilized an egg. In step 40, the machine-learning prediction model analyzes the images of the sperm that was originally selected as well as the input regarding whether or not the sperm successfully fertilized an egg, in order to detect features of the sperm that are indicative of a likelihood of the sperm successfully fertilizing an egg.

Typically, in step 40, the machine-learning prediction model analyzes the data from many procedures in which the preceding steps have been performed, such that the machine-learning prediction model has a large amount of data from which to determine features of the sperm that are indicative of a likelihood of the sperm successfully fertilizing an egg. For some applications, the machine-learning prediction model that is run by the one or more computer processors includes one or more of the following: a neural network (e.g., a convolutional neural network), a Linear Regression, Logistic Regression, Decision Tree, Support Vector Machine, Naive Bayes, kNN, K-Means, Random Forest, Dimensionality Reduction Algorithms, and/or Gradient Boosting algorithm. Typically the images that are acquired are stationary images and the machine-learning prediction model extracts morphological features of the sperm (such as dimensions of respective portions of the sperm, head size, tail size, etc.) and determines morphological features of the sperm that are indicative of a likelihood of the sperm successfully fertilizing an egg. Alternatively or additionally, the images that are acquired are video images and the machine-learning prediction model extracts motility-related features of the sperm, and determines motility-related features of the sperm that are indicative of a likelihood of the sperm successfully fertilizing an egg. It is noted that for some applications, step 40 is performed without the use of a machine-learning prediction model. Rather, for some applications, a different sort of computer-based and/or human-based analysis is applied in order to determine features of the sperm that are indicative of a likelihood of the sperm successfully fertilizing an egg. For such applications, other steps of the procedure that are described herein would vary accordingly.

Typically, in step 42, the one or more computer processors store the output of step 40 for use during the clinical-application stage, as described hereinbelow with reference to FIG. 2B. It is noted that steps 30-42 as described with reference to FIG. 2A are typically performed in an ongoing manner. That is to say that even when the clinical-application stage is performed, it is typically the case that in at least some procedures, the steps described with reference to FIG. 2A are also performed. In this manner, the machine-learning prediction model continues to accrue further data and to further refine features that are identified in the data. It is further noted that the features that are identified within the data do not necessarily have any relationship with the WHO criteria for identifying sperm. Rather, in accordance with the above description, the machine-learning prediction model is configured to identify features of the sperm that are indicative of a likelihood of the sperm successfully fertilizing an egg, based on real world data regarding sperm that did actually successfully fertilize an egg or did not.

Reference is now made to FIG. 2B which is a flowchart showing steps of a sperm-selection optimization method that is performed during ICSI procedures during a clinical-application stage, in accordance with some applications of the present invention. Typically, in a first step 50, a healthcare professional (e.g., the embryologist) views the sperm via a microscope, selects a sperm in accordance with their usual criteria, and then immobilizes the sperm by touching the sperm with the pipette. In parallel with step 50, one or more images of the selected sperm are acquired, in step 52. For some applications, in order to facilitate acquisition of the images that are acquired in step 52, the microscope includes an auxiliary optical output, as described hereinabove. The images that are acquired in step 52 are typically sent to a computer processor. Typically, the images that are acquired in step 52 are of a higher quality than the images that are shown to the embryologist, and the auxiliary optical output has a higher resolution than that of the first optical output (via which the images are shown to the embryologist in real time). As described above, step 52 is typically performed in parallel with step 50. Further typically, the acquisition of the images in step 52 is performed in such a manner that the usual workflow of the embryologist is unchanged and undisturbed.

Typically, both the first optical output (via which the images are shown to the embryologist in real time) and the auxiliary optical output (via which images are outputted for further analysis by a computer processor) receive (and output) optical images that are acquired using cameras, e.g., CMOS cameras. For some applications, both of the cameras receive (and output) images within the visible light spectral range. For some applications, the first optical output receives (and outputs) images within the visible light spectral range and the auxiliary optical output receives (and outputs) images in an alternative or an additional spectral range, e.g., in the short wave infrared (SWIR) spectral range.

In step 54, the images that are acquired in step 52 are received by a computer processor, for comparison with the features of sperm that were identified in the training stage as being indicative of whether a sperm is likely to fertilize an egg. Based on the comparison, the computer processor determines a likelihood of the sperm that was selected in step 50 successfully fertilizing an egg. In step 56, the computer processor generates an output to the embryologist indicating a likelihood of the sperm that they selected in step 50 successfully fertilizing an egg, which is indicative of whether the sperm is a good candidate for the remainder of the ICSI procedure. For example, the computer processor generates an audio output (e.g., via a speaker), or a visual output (e.g., by displaying a score indicating a likelihood that the selected sperm will be a good candidate for the remainder of the ICSI procedure on the image of the sperm that the embryologist is looking at).

It is noted that, typically, the computer processor does not automatically select a sperm or even automatically identify a sperm for selection. Rather, as described above, the embryologist typically selects a sperm in their usual manner, and the computer processor either validates or invalidates the selection. Alternatively, for some applications, the computer processor does automatically identify a sperm for selection and/or automatically selects the sperm. Typically, in response to the computer processor indicating that the selected sperm is a good candidate for the remainder of the ICSI procedure, the embryologist continues to perform the remainder of the ICSI procedure with the selected sperm, in step 58. Further typically, in response to the computer processor indicating that the selected sperm is not a good candidate for the remainder of the ICSI procedure, steps 50-56 are performed again with a different sperm.

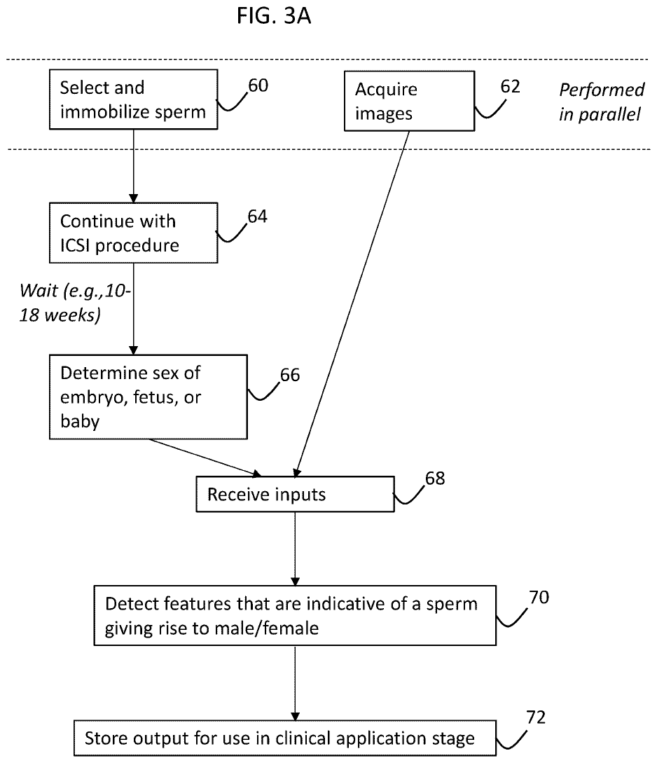

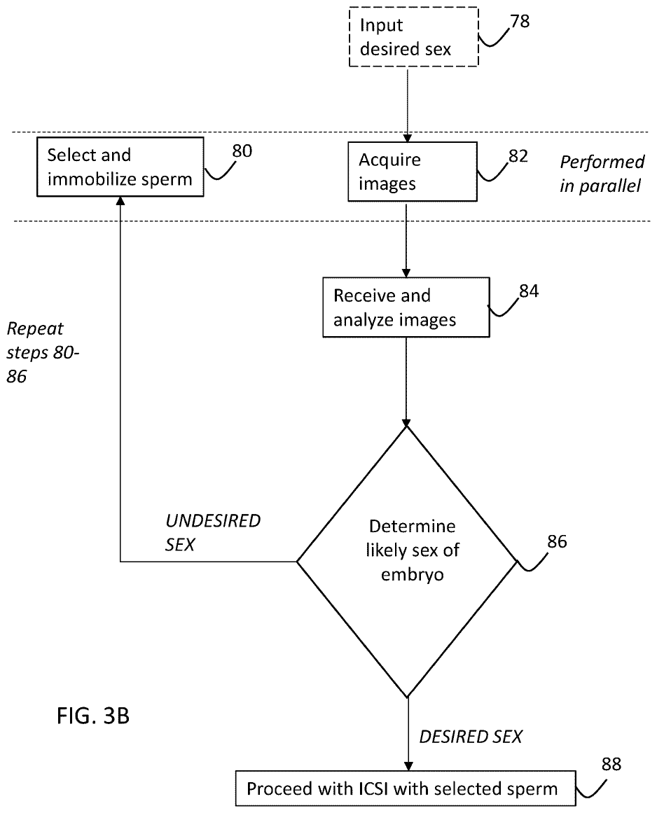

Reference is now made to FIG. 3A, which is a flowchart showing steps of a sex-selection method that is performed during ICSI procedures, during a training stage, in accordance with some applications of the present invention. Typically in a first step 60, the embryologist views the sperm via a microscope, selects a sperm in accordance with their usual criteria, and then immobilizes the sperm by touching the sperm with the pipette. In parallel with step 60, one or more images of the selected sperm are acquired, in step 62. For some applications, in order to acquire the images in step 62, the microscope includes an auxiliary optical output (in addition to the basic auxiliary output via which the images are shown to the embryologist in real time). The images that are acquired in step 62 are typically sent to a computer processor for further analysis as described in further detail hereinbelow. Typically, the images that are acquired in step 62 are of a higher quality than the images that are shown to the embryologist, and the auxiliary optical output has a higher resolution than that of the first optical output (via which the images are shown to the embryologist in real time). As described above, step 62 is typically performed in parallel with step 60. Further typically, the acquisition of the images in step 62 is performed in such a manner that the usual workflow of the embryologist is unchanged and undisturbed.

Typically, both the first optical output (via which the images are shown to the embryologist in real time) and the auxiliary optical output (via which images are outputted for further analysis by a computer processor) receive (and output) optical images that are acquired using cameras, e.g., CMOS cameras. For some applications, both of the cameras receive (and output) images within the visible light spectral range. For some applications, the first optical output receives (and outputs) images within the visible light spectral range and the auxiliary optical output receives (and outputs) images in an alternative or an additional spectral range, e.g., in the short wave infrared (SWIR) spectral range.

In step 64, the embryologist continues with the ICSI procedure in the usual manner, by aspirating the sperm using the pipette, before injecting the aspirated sperm into the cytoplasm of an egg, while the egg is held by a holding tool. In step 66, at a given time period after the fertilization of the egg at which it is possible to determine the sex of the resultant embryo, fetus, or baby the sex of the resultant embryo, fetus, or baby is determined. Typically, the embryologist or a different healthcare professional analyzes the embryo, fetus, or baby to determine the sex of the embryo and inputs this information into a computer processor. For example, the sex of the embryo is determined using preimplantation genetic testing, or it is determined via an ultrasound scan that is performed on the carrier of the embryo or fetus. For some applications, the sex of the resultant embryo or fetus is determined by performing amniocentesis or a placenta biopsy. For some applications, the sex of the resultant fetus is determined between 10 and 18 weeks (e.g., approximately 16 weeks) after the egg has been fertilized, typically by performing an ultrasound scan. For some applications, the sex determination is only performed after the baby is born. The scope of the present disclosure includes determining the sex of the resulting embryo, fetus, or baby by any means, whether during the pregnancy or after the baby is born.

In step 68, the data regarding the sex of the embryo, fetus, or baby, together with the images that were acquired in step 62, are fed as inputs into a machine-learning prediction model that is run by one or more computer processors (which are typically in communication with each other via a network, e.g., computer processors 94 and 96 described hereinbelow with reference to FIG. 4). Thus, the machine-learning prediction model receives both images of the sperm that was originally selected as well as data regarding the sex of the resultant embryo, fetus, or baby. In step 70, the machine-learning prediction model analyzes the images of the sperm that was originally selected as well as the input regarding the sex of the resultant embryo, fetus, or baby in order to detect features of the sperm that are indicative of a likelihood of the sperm giving rise to a male or female embryo.

Typically, in step 70, the machine-learning prediction model analyzes the data from many procedures in which the preceding steps have been performed, such that the machine-learning prediction model has a large amount of data from which to determine features of the sperm that are indicative of a likelihood of the sperm giving rise to a male or female embryo. For some applications, the machine-learning prediction model that is run by the one or more computer processors includes one or more of the following: a neural network (e.g., a convolutional neural network), a Linear Regression, Logistic Regression, Decision Tree, Support Vector Machine, Naive Bayes, kNN, K-Means, Random Forest, Dimensionality Reduction Algorithms, and/or Gradient Boosting algorithm. Typically the images that are acquired are stationary images and the machine-learning prediction model extracts morphological features of the sperm (such as dimensions of respective portions of the sperm, head size, tail size, etc.) and determines morphological features of the sperm that are indicative of a likelihood of the sperm giving rise to a male or female embryo. Alternatively or additionally, the images that are acquired are video images and the machine-learning prediction model extracts motility-related features of the sperm, and determines motility-related features of the sperm that are indicative of a likelihood of the sperm giving rise to a male or female embryo. It is noted that for some applications, step 70 is performed without the use of a machine-learning prediction model. Rather, for some applications, a different sort of computer-based and/or human-based analysis is applied in order to determine features of sperm that are indicative of a likelihood of the sperm giving rise to a male or female embryo. For such applications, other steps of the procedure that are described herein would vary accordingly.

Typically, in step 72, the one or more computer processors store the output of the step 70 for use during the clinical-application stage, as described hereinbelow with reference to FIG. 3B. It is noted that steps 60-72 as described with reference to FIG. 3A are typically performed in an ongoing manner. That is to say that even when the clinical-application stage is performed, it is typically the case that in at least some procedures, the steps described with reference to FIG. 3A are also performed. In this manner, the machine-learning prediction model continues to accrue further data and to further refine features that are identified in the data.

Reference is now made to FIG. 3B which is a flowchart showing steps of a sex-selection optimization method that is performed during ICSI procedures during a clinical-application stage, in accordance with some applications of the present invention. For some applications, in a first step 78, the embryologist inputs whether a male or a female baby is desired (e.g., based on input from the prospective parent(s)). (Step 78 is in a dashed box to indicate that it is optional.) Typically, in step 80, the embryologist views the sperm via a microscope, selects a sperm in accordance with their usual criteria, and then immobilizes the sperm by touching the sperm with the pipette. In parallel with step 80, one or more images of the selected sperm are acquired, in step 82. For some applications, images are acquired in step 82 using an auxiliary optical output, as described hereinabove. The images that are acquired in step 82 are typically sent to a computer processor. Typically, the images that are acquired in step 82 are of a higher quality than the images that are shown to the embryologist, and the auxiliary optical output has a higher resolution than that of the first optical output (via which the images are shown to the embryologist in real time). As described above, step 82 is typically performed in parallel with step 80. Further typically, the acquisition of the images in step 82 is performed in such a manner that the usual workflow of the embryologist is unchanged and undisturbed.

Typically, both the first optical output (via which the images are shown to the embryologist in real time) and the auxiliary optical output (via which images are outputted for further analysis by a computer processor) receive (and output) optical images that are acquired using cameras, e.g., CMOS cameras. For some applications, both of the cameras receive (and output) images within the visible light spectral range. For some applications, the first optical output receives (and outputs) images within the visible light spectral range and the auxiliary optical output receives (and outputs) images in an alternative or an additional spectral range, e.g., in the short wave infrared (SWIR) spectral range.

In step 84, the images that are acquired in step 82 are sent to a computer processor, for comparison with the features of the sperm that were identified in the training stage as being indicative of a likelihood of the sperm giving rise to a male or female embryo. Based on the comparison, the computer processor determines a likelihood of the sperm that was selected in step 80 giving rise to a male or a female embryo. In step 86, the computer processor generates an output to the embryologist indicating the likelihood of the sperm that they selected in step 80 giving rise to a male or a female embryo. For example, the computer processor generates an audio output (e.g., via a speaker), or a visual output (e.g., by generating an output on the image of the sperm that the embryologist is looking at, e.g., a likelihood of the sperm giving rise to an embryo of a given sex). As described above, for some applications, in a first step 78, the embryologist inputs whether a male or a female baby is desired. For some such applications, an output is generated that indicates that the embryologist should proceed with the sperm that was selected in step 80 or that they should select a new sperm. Typically, in cases in which the sperm that was selected in step 80 has been determined as being likely to give rise to an embryo of the opposite sex to the desired sex, then steps 80-86 are repeated.

For some applications, during the clinical-application stage, both the steps that are described with reference to FIG. 2B as well as the steps that are described with reference to FIG. 3B are performed. In other words, the computer processor analyzes the sperm that is selected by the embryologist both to determine whether the sperm is likely to successfully fertilize an egg and also to determine the likely sex of the resulting embryo. Typically, for such applications, the embryologist repeats the process of selecting sperm until the computer processor generates an output indicating both that the sperm is likely to successfully fertilize an egg and that the likely sex of the resulting embryo is the desired sex.

For some applications, generally similar steps to those described with reference to FIGS. 2A and 2B are performed. However, as an alternative to, or in addition to, the steps described hereinabove, in the training stage, at the appropriate time, an input is provided to the machine-learning prediction model indicating whether the fetus that developed from the ICSI procedure that was performed with the selected sperm was viable or not, whether a baby was born or not, whether the baby was healthy or not, and/or any medical conditions that the baby has. For example, such an input may be provided once the baby was born, or after 5-8 months once it becomes clear whether the fetus is viable and/or whether the fetus has any medical conditions. Using generally similar techniques to those described hereinabove mutatis mutandis, the machine-learning prediction model analyzes the images of the sperm that was originally selected as well as the above-described inputs, in order to detect features of the sperm that are indicative of sperm being likely to give rise to a viable or non-viable fetus, to give rise to a baby being born or not, to give rise to a healthy or an unhealthy baby, and/or to give rise to a baby having a given any medical condition. (For some applications, a different sort of computer-based and/or human-based analysis is applied in order to determine features of sperm that are indicative of sperm being likely to give rise to a viable or non-viable fetus, to give rise to a baby being born or not, to give rise to a healthy or an unhealthy baby, and/or to give rise to a baby having a given any medical condition.) Subsequently, in the clinical-application stage, in response to the embryologist selecting and immobilizing a given sperm, the computer processor determines a likelihood of the sperm giving rise to a viable or non-viable fetus, giving rise to a baby being born or not, giving rise to a healthy or an unhealthy baby, and/or giving rise to a baby having a given any medical condition, and generates an output accordingly.

For some applications, generally similar techniques to those described hereinabove are performed with respect to eggs that are selected for an IVF treatment (e.g., for an ICSI procedure of an IVF treatment). For some such application generally similar steps to those described with reference to FIGS. 2A-B are performed with reference to an eggs rather than sperm. Typically, during the training stage, features of eggs (e.g., morphological features of eggs) that are indicative of eggs giving rise to a successful fertilization are determined. Subsequently, in the clinical-application stage, in response to an egg being selected for use in the IVF treatment, the computer processor generates an output to the embryologist indicating a likelihood of the selected egg being successfully fertilized. For some applications, the machine-learning prediction model is trained with respect to combinations of sperm and eggs, such that it is trained to identify which combinations of features of sperm and eggs are likely to cause an egg to be successfully fertilized. Subsequently, during the clinical-application stage, the computer processor indicates a likelihood of a selected sperm successfully fertilizing a given egg, and/or vice versa (i.e., the computer processor indicates a likelihood of a selected egg being successfully fertilized by a given sperm). For some applications, generally similar techniques to those described hereinabove are performed with respect to an egg, in order to determine a likelihood of an egg (or a combination of a sperm and an egg) giving rise to a viable or non-viable fetus, giving rise to a born baby, giving rise to a healthy baby, and/or giving rise to a baby having one or more medical conditions.

Reference is now made to FIG. 4, which is a block diagram showing an example of a microscope 90 with an auxiliary optical output 92, in accordance with some applications of the present invention. As described hereinabove, for some applications, a healthcare professional (e.g., the embryologist) views the sperm via a first optical output 91 of microscope 90 (which is typically an in-built microscope display), selects a sperm in accordance with their usual criteria, and then immobilizes the sperm by touching the sperm with the pipette. In parallel with the aforementioned step, one or more images of the selected sperm are acquired for analysis by one or more computer processors, e.g., a local computer processor 94 and/or one or more additional computer processors 96 that are typically remote from the local computer processor and are connected to the local computer processor via a network. For some applications, in order to facilitate acquisition of the images that are acquired for analysis by the computer processor(s), the microscope includes an auxiliary optical output (in addition to the first optical output via which the images are shown to the embryologist in real time). Typically, the images that are analyzed by the computer processor(s) are of a higher quality than the images that are shown to the embryologist, and the auxiliary optical output has a higher resolution than that of the first optical output (via which the images are shown to the embryologist in real time). For example, the magnification of the first optical output may be between 150 and 450 (e.g., 200 plus/minus 50, or 400 plus/minus 50) and that of the auxiliary optical output may be between 350 and 850 (e.g., 400 plus/minus 50, or 800 plus/minus 50). For some applications, a ratio of the magnification of the auxiliary output to that of the first optical output is at least 3:2 e.g., at least 2:1. As described above, typically, the acquisition of the images by the auxiliary optical output is performed in such a manner that the usual workflow of the embryologist is unchanged and undisturbed.

Typically, both the first optical output (via which the images are shown to the embryologist in real time) and the auxiliary optical output (via which images are outputted for further analysis by a computer processor) receive (and output) optical images that are acquired using cameras, e.g., CMOS cameras. For some applications, both of the cameras receive (and output) images within the visible light spectral range. For some applications, the first optical output receives (and outputs) images within the visible light spectral range and the auxiliary optical output receives (and outputs) images in an alternative or an additional spectral range, e.g., in the short wave infrared (SWIR) spectral range.

As described hereinabove, in accordance with some applications of the present invention, algorithms are applied in order to determine a likelihood of sperm, eggs, and/or combinations of sperm and eggs giving rise to a successful fertilization, an embryo of a desired sex, a viable or non-viable fetus, a baby being born or not, a healthy or an unhealthy baby, and/or a baby having a given any medical condition. For some applications, once such features are determined with respect to a set of data that was acquired under a given set of conditions, e.g., at a given hospital, within a given city, within a given country, among a given demographic group, a transfer-learning algorithm is applied in order to apply such prediction techniques to data that are acquired under a different set of conditions, e.g., at a different hospital, within a different city, within a different country, and/or among a different demographic group. It is noted that typically transfer learning techniques are applied in order to transfer knowledge that is gained with respect to determining a first outcome (e.g., recognizing cats) to determining a different outcome (e.g., recognizing dogs). In the case of the present disclosure the same outcome is being determined (e.g., a likelihood of sperm, eggs, and/or combinations of sperm and eggs giving rise to a successful fertilization, an embryo of a desired sex, a viable or non-viable fetus, a baby being born or not, a healthy or an unhealthy baby, and/or a baby having a given any medical condition) but with respect to datasets that are acquired under different conditions from each other (with such conditions typically being likely to impact the relevant features for the determination). For example, different hospitals (and certainly hospitals in different cities or countries from each other) are likely to use different types of equipment, and to cater to different demographic groups (who typically have different genetic makeups).

Applications of the invention described herein can take the form of a computer program product accessible from a computer-usable or computer-readable medium (e.g., a non-transitory computer-readable medium) providing program code for use by or in connection with a computer or any instruction execution system, such as computer processors 94 and 96. For the purpose of this description, a computer-usable or computer readable medium can be any apparatus that can comprise, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Typically, the computer-usable or computer readable medium is a non-transitory computer-usable or computer readable medium.

Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W), DVD, and a USB drive.

A data processing system suitable for storing and/or executing program code will include at least one processor (e.g., computer processor 94 or 96) coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution. The system can read the inventive instructions on the program storage devices and follow these instructions to execute the methodology of the embodiments of the invention.

Network adapters may be coupled to the processor to enable the processor to become coupled to other processors or remote printers or storage devices through intervening private or public networks. Modems, cable modem and Ethernet cards are just a few of the currently available types of network adapters.

Computer program code for carrying out operations of the present invention may be written in any combination of one or more programming languages, including an object-oriented programming language such as Java, Smalltalk, C++ or the like and conventional procedural programming languages, such as the C programming language or similar programming languages.

It will be understood that the algorithms described herein, can be implemented by computer program instructions. These computer program instructions may be provided to a processor of a general-purpose computer, special purpose computer, or other programmable data processing apparatus to produce a machine, such that the instructions, which execute via the processor of the computer (e.g., computer processor 94 or 96) or other programmable data processing apparatus, create means for implementing the functions/acts specified in the algorithms described in the present application. These computer program instructions may also be stored in a computer-readable medium (e.g., a non-transitory computer-readable medium) that can direct a computer or other programmable data processing apparatus to function in a particular manner, such that the instructions stored in the computer-readable medium produce an article of manufacture including instruction means which implement the function/act specified in the algorithms. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide processes for implementing the functions/acts specified in the algorithms described in the present application.

Computer processors 94 and 96 are typically hardware devices programmed with computer program instructions to produce a special purpose computer. For example, when programmed to perform the algorithms described with reference to the Figures, computer processors 94 and 96 typically act as special purpose IVF-analysis computer processors. Typically, the operations described herein that are performed by computer processors 94 and 96 transform the physical state of a memory, which is a real physical article, to have a different magnetic polarity, electrical charge, or the like depending on the technology of the memory that is used. For some applications, operations that are described as being performed by a computer processor are performed by a plurality of computer processors (e.g., computer processors 94 and 96) in combination with each other.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. An apparatus, comprising:
one or more computer processors configured:
during a training stage:
to receive one or more images of sperm that are selected for use in ICSI procedures, the images being acquired at the same time as the sperm for respective ICSI procedures are selected by a healthcare professional,
subsequently, receive inputs that are indicative of whether sperm that were selected for respective ICSI procedures gave rise to male or female embryos, and
analyze the images and the inputs that are indicative of whether sperm that were selected for respective ICSI procedures gave rise to male or female embryos, to identify features of sperm that are indicative of likelihoods of the sperm giving rise to male or female embryos.

2-7. (canceled)

8. The apparatus according to claim 1, wherein the one or more computer processors are configured to apply a transfer-learning algorithm to the identified features in order to:
analyze data relating to an additional set of sperm that were acquired under a different set of conditions from the sperm in the one more images, and
identify features in the data relating to the additional set of sperm that are indicative of likelihoods of sperm within the additional set of sperm giving rise to male or female embryos.

9. The apparatus according to claim 1, wherein the one or more computer processors are configured to receive one or more stationary images of the sperm that are selected for use in ICSI procedures and to identify morphological features of the sperm that are indicative of likelihoods of the sperm giving rise to male or female embryos by analyzing the one or more stationary images of the sperm.

10. (canceled)

11. The apparatus according to claim 1, wherein the one or more computer processors are further configured, during a clinical-application stage, to:
receive one or more images of a sperm that is selected for use in a current ICSI procedure, the images being acquired at the same time as the sperm for the current ICSI procedures is selected by a healthcare professional,
determine a likelihood of the selected sperm giving rise to a male or a female embryo by comparing features of the selected sperm to the features that were identified in the training stage, and
generate an output that is indicative of the likelihood of the selected sperm giving rise to a male or a female embryo.

12-15. (canceled)

16. A method comprising:
using one or more computer processors, during a training stage:
receiving one or more images of sperm that are selected for use in ICSI procedures, the images being acquired at the same time as the sperm for respective ICSI procedures are selected by a healthcare professional;
subsequently, receiving inputs that are indicative of whether sperm that were selected for respective ICSI procedures gave rise to male or female embryos; and
analyzing the images and the inputs that are indicative of whether sperm that were selected for respective ICSI procedures gave rise to male or female embryos, to identify features of sperm that are indicative of likelihoods of the sperm giving rise to male or female embryos.

17. The method according to claim 16, wherein receiving inputs that are indicative of whether sperm that were selected for respective ICSI procedures gave rise to male or female embryos comprises receiving inputs based on preimplantation genetic testing performed on at least some embryos resulting from the ICSI procedures.

18. The method according to claim 16, wherein receiving inputs that are indicative of whether sperm that were selected for respective ICSI procedures gave rise to male or female embryos comprises receiving inputs based on ultrasound scans performed on at least some embryos or fetuses resulting from the ICSI procedures.

19. The method according to claim 16, wherein receiving inputs that are indicative of whether sperm that were selected for respective ICSI procedures gave rise to male or female embryos comprises receiving inputs based on amniocentesis performed on carriers of at least some of the embryos resulting from the ICSI procedures.

20. The method according to claim 16, wherein receiving inputs that are indicative of whether sperm that were selected for respective ICSI procedures gave rise to male or female embryos comprises receiving inputs based on placenta biopsies performed on carriers of at least some of the embryos resulting from the ICSI procedures.

21. The method according to claim 16, wherein receiving inputs that are indicative of whether sperm that were selected for respective ICSI procedures gave rise to male or female embryos comprises receiving inputs indicating whether born babies resulting from the ICSI procedures were male or female.

22. The method according to claim 16, wherein receiving inputs that are indicative of whether sperm that were selected for respective ICSI procedures gave rise to male or female embryos comprises receiving the inputs 10-18 weeks after the respective ICSI procedures.

23. The method according to claim 16, further comprising applying a transfer-learning algorithm to the identified features in order to:
analyze data relating to an additional set of sperm that were acquired under a different set of conditions from the sperm in the one more images, and
identify features in the data relating to the additional set of sperm that are indicative of likelihoods of sperm within the additional set of sperm giving rise to male or female embryos.

24. The method according to claim 16, wherein receiving one or more images of sperm that are selected for use in ICSI procedures comprises receiving one or more stationary images of the sperm that are selected for use in ICSI procedures, the method comprising identifying morphological features of the sperm that are indicative of likelihoods of the sperm giving rise to male or female embryos by analyzing the one or more stationary images of the sperm.

25. The method according to claim 16, wherein receiving one or more images of sperm that are selected for use in ICSI procedures comprises receiving one or more video images of the sperm that are selected for use in ICSI procedures, the method comprising identifying motility-related features of the sperm that are indicative of likelihoods of the sperm giving rise to male or female embryos by analyzing the one or more video images of the sperm.

26. The method according to claim 16, further comprising, during a clinical-application stage:
receiving one or more images of a sperm that is selected for use in a current ICSI procedure, the images being acquired at the same time as the sperm for the current ICSI procedures is selected by a healthcare professional;
determining a likelihood of the selected sperm giving rise to a male or a female embryo by comparing features of the selected sperm to the features that were identified in the training stage; and
generating an output that is indicative of the likelihood of the selected sperm giving rise to a male or a female embryo.

27. The method according to claim 16, further comprising:
receiving inputs that are indicative of whether sperm that were selected for respective ICSI procedures gave rise to viable fetuses, and
analyzing the images and the inputs that are indicative of whether sperm that were selected for respective ICSI procedures gave rise to viable fetuses, to identify features of sperm that are indicative of likelihoods of the sperm giving rise to viable fetuses.

28. The method according to claim 16, further comprising:
receiving inputs that are indicative of whether sperm that were selected for respective ICSI procedures gave rise to born babies, and
analyzing the images and the inputs that are indicative of whether sperm that were selected for respective ICSI procedures gave rise to born babies, to identify features of sperm that are indicative of likelihoods of the sperm giving rise to born babies.

29. The method according to claim 16, further comprising:
receiving inputs that are indicative of whether sperm that were selected for respective ICSI procedures gave rise to healthy babies, and
analyzing the images and the inputs that are indicative of whether sperm that were selected for respective ICSI procedures gave rise to healthy babies, to identify features of sperm that are indicative of likelihoods of the sperm giving rise to healthy babies.

30. The method according to claim 16, further comprising:
receiving inputs that are indicative of whether sperm that were selected for respective ICSI procedures gave rise to babies having a given medical condition, and
analyzing the images and the inputs that are indicative of whether sperm that were selected for respective ICSI procedures gave rise to babies having the given medical condition, to identify features of sperm that are indicative of likelihoods of the sperm giving rise to babies having the given medical condition.

31. A computer software product comprising a tangible non-transitory computer-readable medium in which program instructions are stored, which instructions, when read by one or more computer processors, cause the one or more computer processors, during a training stage:
to receive one or more images of sperm that are selected for use in ICSI procedures, the images being acquired at the same time as the sperm for respective ICSI procedures are selected by a healthcare professional,
subsequently, receive inputs that are indicative of whether sperm that were selected for respective ICSI procedures gave rise to male or female embryos, and
analyze the images and the inputs that are indicative of whether sperm that were selected for respective ICSI procedures gave rise to male or female embryos, to identify features of sperm that are indicative of likelihoods of the sperm giving rise to male or female embryos.

32-87. (canceled)
